(19) United States
(12) Patent Application Publication
(10) Pub. No.: US 2026/0114998 A1
(43) Pub. Date: Apr. 30, 2026

(54) Cardiac Clips

(71) Applicant: v2vmedtech, Inc., Maple Grove, MN (US)

(72) Inventors: Michael B. McDonald, Cordova, TN (US); Vinayak N. Bapat, Minneapolis, MN (US)

(21) Appl. No.: 19/479,144

(22) PCT Filed: May 30, 2024
(86) PCT No.: PCT/US2024/031627

§ 371 Date: Oct. 27, 2025

Related U.S. Application Data
(60) Provisional application No. 63/469,923, filed on May 31, 2023.

(51) Int. Cl. A61F 2/24

(52) U.S. Cl. CPC A61F 2/2466; A61F 2/2454

(57) ABSTRACT

Cardiac conditions such as valvular regurgitation can be treated using implantable clip devices. Some clip devices include two or more separate clips that, after individual attachment to native valve leaflet portions, are then coupled to each other. The finished arrangement can serve to maintain leaflet portions that are held in or near coaptation with each other by the clips.

### CROSS-REFERENCE TO RELATED APPLICATIONS

This application claims the benefit of U.S. Provisional Application Ser. No. 63/469,923 filed May 31, 2023. The disclosure of the prior application is considered part of (and is incorporated by reference in) the disclosure of this application.

### BACKGROUND

The disclosure relates to medical devices and methods of surgery. More particularly, the invention relates to clips implantable in the heart of a patient.

The mitral valve maintains unidirectional blood flow between the left atrium and the left ventricle. The tricuspid valve maintains unidirectional blood flow between the right atrium and right ventricle. Mitral and tricuspid regurgitation will occur when the valve leaflets do not coapt (seal) properly. Valvular regurgitation can often lead to heart failure and, in some cases, death.

Transcatheter correction of mitral and tricuspid regurgitation by implantation of a clip (MitraClip from Abbott and PASCAL from Edwards Lifesciences) has become standard therapy for patients at high risk for open heart surgical corrective procedures. Such clip implantation procedures are performed through a guiding catheter that is inserted into the femoral vein. One or more clips can be delivered through the guiding catheter and implanted to reapproximate the valve leaflets. This procedure is often referred to as “edge-to-edge” repair.

The present clips are a single structure with four clip arms extending out at a 180-degree angle. One of the limitations to the “edge-to-edge” repair is being able to reach both of the leaflets with the clip. At times, the valve leaflets are too far apart to be simultaneously reached by both arms of the clip. Another limitation of the “edge-to-edge” repair is that when the present clips are not placed exactly perpendicular to the valve plane opening, the present clips can torque the leaflets of the valve. This torquing of the leaflets creates a situation called “pinwheeling” of the valve. This “pinwheeling” can cause problems, such as residual valve regurgitation and possible clip detachment.

### BRIEF DESCRIPTION OF THE DRAWINGS

For a more detailed description of the embodiments of the present disclosure, reference will now be made to the accompanying drawings, wherein:

FIG. 1 is a schematic view of a clip in accordance with an embodiment, wherein the clip is screwed to a delivery sheath and entirely housed in a small bore steerable guide;

FIG. 2 is a schematic view of the clip shown in FIG. 1, wherein the clip is advanced out of the small bore steerable guide, and a main arm of the clip deploys;

FIG. 3 is a schematic view of the clip shown in FIG. 1, wherein a tether wire is partially released, allowing a gripper arm of the clip to deploy;

FIG. 4 is a schematic view of the clip shown in FIG. 1, wherein the tether wire is further released, allowing the gripper arm to close against the main arm;

FIG. 5 is a schematic view of two clips as shown in FIG. 1, wherein the two clips are joined in a parallel configuration by a coupling collar;

FIG. 6 is a schematic view of two clips adapted to be joined in a crossed configuration and their associated delivery sheaths in accordance with an embodiment, wherein a coupling collar is being advanced over the associated delivery sheaths using a collar delivery catheter;

FIG. 7 is a schematic view of the two clips shown in FIG. 6, wherein the coupling collar joins the two clips;

FIG. 8 is a schematic view of two clips adapted to be joined in a parallel configuration and their associated delivery sheaths in accordance with an embodiment, wherein the main arm and the gripper arm have different rest positions;

FIG. 9 is a schematic view of the two clips shown in FIG. 8, wherein the coupling collar joins the two clips;

FIG. 10 is a schematic view of two clips attached to their delivery shafts, where the clips have outward flares;

FIGS. 11 and 12 are schematic views of the two clips shown in FIG. 10, each provided in a delivery sheath having a tether wire or gripper line, and both delivery sheaths provided a lumen of a steerable guide;

FIGS. 13 and 14 are schematic views of the two clips shown in FIG. 10, illustrating the effect or sequentially releasing the tension of the tether wires;

FIG. 15 is a schematic view of the two clips shown in FIG. 10, illustrating the removal of both delivery sheaths and tether wires or gripper lines;

FIGS. 16 and 17 are schematic views of the two clips shown in FIG. 10, illustrating the control of the degree of the outward flare of the clips using the steerable guide; and

FIG. 18 is a schematic view of the two clips shown in FIG. 10, with a coupling collar having a skirt locked in place before the retrieval of the clip delivery shafts.

As is customary, the drawings may not be drawn to scale for the sake of clarity.

### DETAILED DESCRIPTION

It is to be understood that the following disclosure describes several exemplary embodiments for implementing different features, structures, or functions of the invention. Exemplary embodiments of components, arrangements, and configurations are described below to simplify the disclosure; however, these exemplary embodiments are provided merely as examples and are not intended to limit the scope of the invention.

### IMPLANT

The disclosure describes a preferred embodiment of an implant that comprises two or more clips. Each clip comprises a shaft, a single main arm, which is relatively more rigid, and a single gripper arm, which is relatively more flexible and is located above the main arm. The main arm and/or the gripper arm are attached to the shaft via a hinge area. Each clip can be moved independently from the other clips and grasp a valve leaflet. Once a proper grasp of the leaflets is completed with all the clips, the clips can be joined together, thus forming a single structure. This implant may also allow the clips to separate at a later date if needed.

For example, the implant also comprises a coupling collar (e.g., a stent, a tube) that is movable along the clips toward the main arm/gripper arm. The coupling collar may contain two halves joined together in a locking fashion and a central lumen. The distal end of the coupling collar may have a skirt, funnel, or umbrella that flares out over the atrial surface of the cardiac valve as the coupling collar moves toward the cardiac valve. By pulling the clips together with the coupling collar, the valve leaflets are joined together. Locking grooves or ridges are present on the proximal end of each shaft. These locking grooves or ridges can secure the coupling collar at the proper level, joining the two or more clips. For example, the coupling collar includes complementary locking grooves or ridges that can interlock with the locking grooves or ridges on each shaft. The coupling collar can join the two or more clips together either in a parallel or crossed configuration. The coupling collar can optionally have a central lumen. In order to separate previously joined clips, a coronary angioplasty balloon can be positioned in the central lumen of the coupling collar. When the balloon is inflated, the two halves of the coupling collar are disjoined, and the clips are separated.

Alternatively, the clips can be joined together or separated using a nut and bolt that can be screwed on and off the clips.

Alternatively yet, the clips may be separated by applying energy such as electric energy to the implant.

The clip is preferably of nitinol construction.

The shaft of the clips can be flattened or faceted on one side to ensure a consistent orientation of the arms of the two or more clips when they are joined. The side that is flattened or faceted depends on whether the two or more clips are joined together in a parallel or crossed configuration. However, the shaft of the clips is preferably rounded. As such, the shaft can rotate inside the coupling collar even when the coupling collar has been moved in place to interlock with the locking grooves or ridges on each shaft. This configuration may alleviate occurrences of “pinwheeling” of the valve.

The resting configuration of the clip is preferably with the arms closed against each other and at a 90-degree angle or less from the vertical shaft of the clip. For example, the clips can be configured with the arms angled up, which may improve tissue bonding.

### DELIVERY KIT

The disclosure describes three preferred embodiments of a delivery kit for implanting the clips. Each clip is releasably connected (e.g., screwed) to a corresponding delivery shaft that is inserted into a delivery sheath that houses it. The delivery sheaths and/or the delivery shafts may have a slight outward angle leading to an outward flare of the clips. Each delivery sheath also houses a tether wire that can be used to raise and lower the gripper arm of the clip. The tether wire runs through the delivery sheath and out to the tip of the gripper arm. The coupling collar can advance over the delivery shafts after the delivery sheaths and tether wires are removed.

In order to have the ability for each clip to reach out enough to grab valve leaflets that do not coapt, the delivery kit can include a plurality of small bore steerable guides-typically two guides, one for each of the clips.

A large bore steerable catheter may house all the clips, their delivery sheaths, and their small bore steerable guides. The combination of two steerable guides (the large bore steerable guide and the small bore steerable guide inside of it) can give a lot of flexibility and control to direct the clips.

After removing the small bore steerable guides from the large bore steerable catheter, the large bore steerable catheter may house a collar delivery catheter at the distal end of which the coupling collar is provided. The collar delivery catheter and the coupling collar surround the delivery shafts.

In an alternative embodiment, the delivery kit may include fewer and sometimes only one small bore steerable guide. A plurality and sometimes all the clips can be contained and delivered by a small bore steerable guide. Typically, the small bore guide may contain two clip delivery shafts. In this case, each of the delivery sheaths and/or the delivery shafts preferably has the slight outward angle leading to the outward flare of the clips.

The collar is delivered inside a small bore steerable guide on the end of the collar delivery catheter (e.g., on the distal tip of the collar delivery catheter). The collar can lock onto the clips as previously described.

In yet another alternative embodiment, the clips may be delivered to the valve by using only the large bore guide. This alternative does not require the small bore guide(s).

### PROCEDURE

The disclosure describes three preferred embodiments of a surgical procedure.

Initially, each clip is connected to the distal end of a delivery shaft, which is inserted in a delivery sheath. Each delivery sheath contains a tether wire for controlling the position of the gripper arm of the clip. The tether wire is looped through a passageway in the delivery sheath and is attached to the distal end of the gripper arm. Each clip and its delivery shaft/sheath are optionally contained in a small bore steerable guide, either individually or collectively. The delivery shafts/sheaths, and if provided, the small bore steerable guide(s), are housed in the large bore steerable catheter.

In the mitral procedure, the large bore steerable catheter is used for crossing the atrial septum and pointing the implant down toward the mitral valve. Thus, the large bore steerable catheter is placed across the atrial septum, pointing down at the mitral valve.

In one embodiment, each clip and the delivery shaft/sheath may be individually housed within a small bore steerable guide. When housed in the small bore steerable guide, the main arm is vertical in a downward position, and the gripper arm is vertical in an upright position.

When positioned at the cardiac valve plane, each individual clip and its corresponding small bore steerable guide is sequentially advanced out of the large bore steerable catheter.

Then, when the first clip is advanced out of the small bore steerable guide, the main arm comes to a 90-degree position. The small bore steerable guide can be used to control the degree of the outward flare of the clips (e.g., flatten the delivery sheath and/or delivery shaft). The main arm is then brought up against the ventricular side of the valve leaflet. The small bore steerable guide is then retracted further, which releases the gripper arm of the clip. The tether wire keeps the gripper arm in a retracted position until the final positioning of the clip. The tether wire is then partially released, which closes the gripper arm against the atrial side of the valve. The tether wire is preferably fully released and retrieved together with the delivery sheath later in the procedure, as is explained below. The other clips are deployed in the same fashion as the first clip.

The clips are repositionable up until the release of the tether wire. Thus, the tether wires are preferably not fully released until the proper positioning of all the clips has been confirmed. Once the tether wires are released, the delivery sheaths can be retrieved from around the delivery shafts.

Once all the clips have been positioned, the small bore steerable guides are removed. This step leaves just the clip delivery shafts in the lumen of the large bore steerable catheter.

Then, the coupling collar is applied, which joins the two or more clips together. The coupling collar is contained at the distal end of a collar delivery catheter. The collar delivery catheter and coupling collar are advanced inside the large bore steerable catheter over the two or more delivery shafts and advanced down to the clips and out of the end of the large bore steerable catheter. By pulling the clips together with the coupling collar, the valve leaflets are joined together or brought close to one another.

The release of the clips may not be complete until the coupling collar is advanced down over the delivery sheaths. Once the coupling collar is in place, the delivery shafts may be released from the clip. Once the clips are joined together by the coupling collar and still contained within the collar delivery catheter, each of the delivery shafts is unscrewed from its clip. The collar and the clips are locked together at this point, and the implant can then be pushed out of the distal tip of the large bore steerable guide. Once unscrewed and still within the collar delivery catheter, these delivery shafts can be advanced against the coupling collar to free it from the collar delivery catheter (e.g., in a plunger-like manner).

At the end of the procedure, the clips are joined, applied to the leaflets, and free of the collar delivery catheter.

In the tricuspid valve procedure, the large bore steerable catheter is just used for the delivery of the implant. The clips may be applied to the tricuspid valve in a similar fashion without the need for septal crossing.

In another embodiment, the clips and their delivery shafts/sheaths may be collectively housed within a small bore steerable guide. When housed in the small bore steerable guide, the main arm of each clip is vertical in a downward position, and the gripper arm is vertical in an upright position. The large bore steerable guide catheter typically stays up in the atrium, and the small bore steerable guide feeds through it.

In this embodiment, the small bore steerable guide(s) may not need to be removed from the large bore steerable catheter. The clip delivery sheath and gripper line may still be removed to allow the delivery of the collar. The collar may slide down over the clip delivery shafts. The collar may go down through the small bore steerable guide. Once the collar is locked on, the collar delivery catheter can be used as a plunger to push the clips and collar out of the small bore guide catheter.

In yet another embodiment, the clips may be delivered to the valve by using only the large bore guide.

If, at a later date, there is a need to separate the clips, a balloon can be used to separate the coupling collar into halves. This action will free the clips. Each clip can remain on the individual valve leaflet.

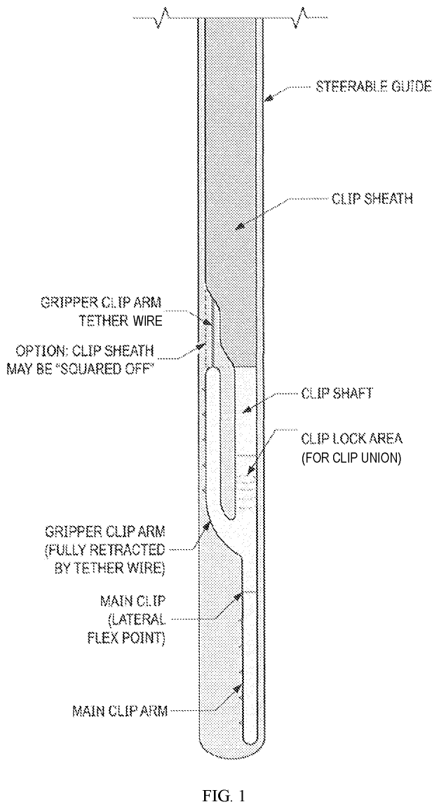

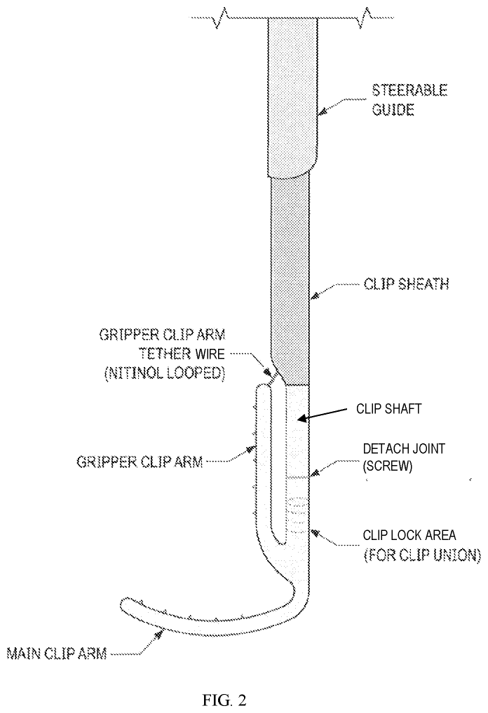

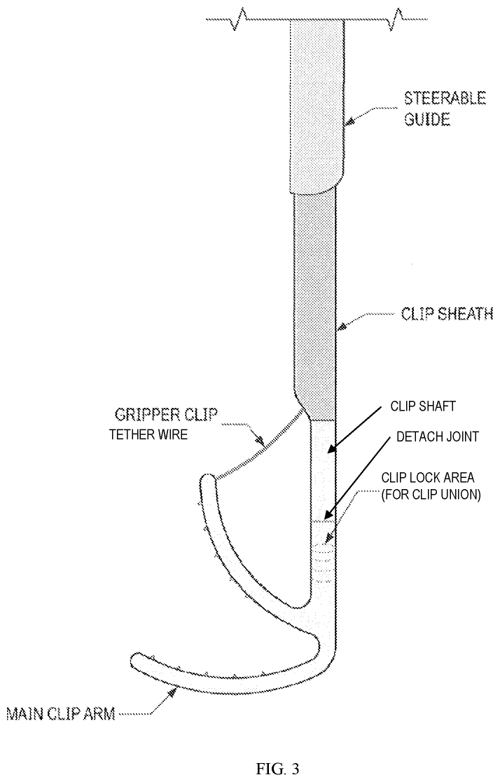

FIGS. 1 to 4 illustrate a sequence of the deployment of a clip. In use, two or more clips are deployed as shown in FIGS. 1 to 4. Each clip can be moved independently from the other clips and grasp a valve leaflet. Once a proper grasp of the leaflets is completed with all the clips, the clips can be joined together, thus forming a single structure.

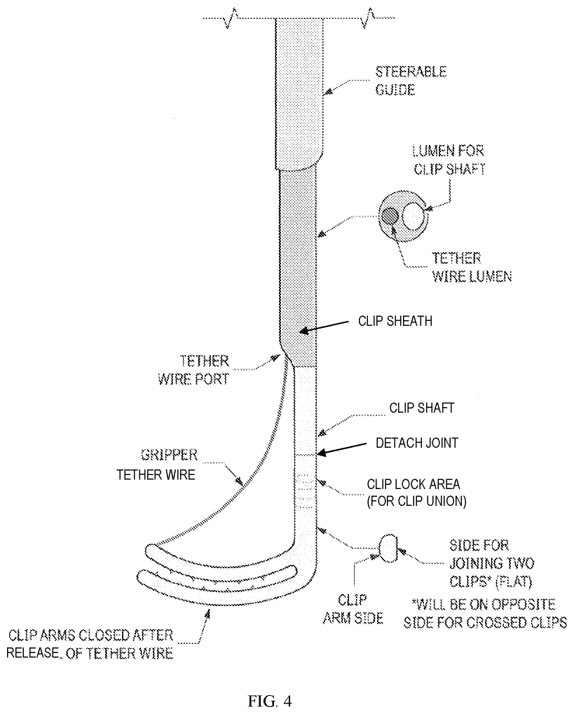

After the step illustrated in FIG. 4, the tether wire and clip delivery sheaths are retrieved.

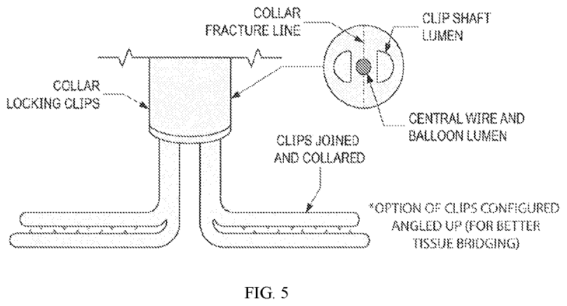

FIG. 5 illustrates two clips deployed as illustrated in FIGS. 1 to 4, and joined by a coupling collar. In this example, the coupling collar contains two halves joined together in a locking fashion and a central lumen. In order to separate previously joined clips, a coronary angioplasty balloon can be positioned in the central lumen of the coupling collar. When the balloon is inflated, the two halves of the coupling collar are disjoined, and the clips are separated.

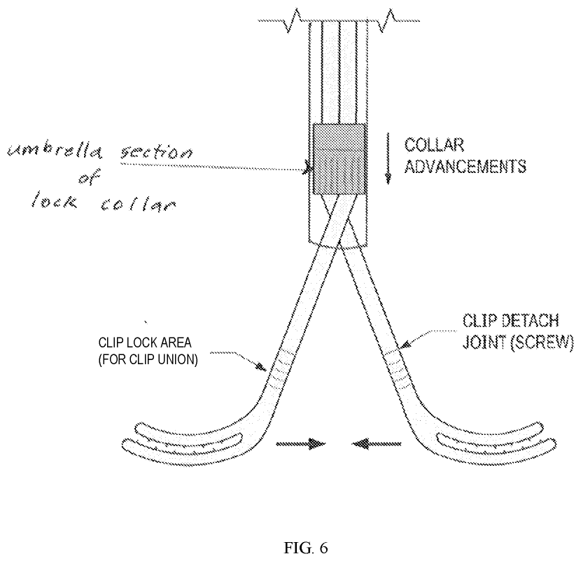

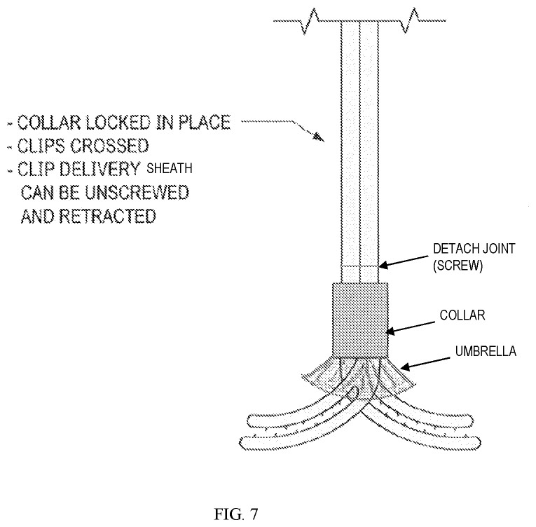

FIGS. 6 and 7 illustrate an example sequence of joining two clips that have clip shafts that are arranged relative to each other in a crossed configuration. Each clip shaft has a natural non-linear shape. The clip shafts are arranged opposite of each other (e.g., about 180° opposite of each other) so that they extend radially outward away from each other when unconstrained, as shown in FIG. 6.

Each clip shaft can be made of an elastically-flexible material (e.g., a Nitinol tube) that is shape-set to have a naturally curved or angled shape. In result, the clip shafts (and clips attached at a distal end thereof) have a natural tendency to splay radially outward when unconstrained by the sheath and/or collar (as depicted in FIG. 6). However, by sliding the collar and/or sheath distally to capture the curved portion(s) of the clip shafts therein, the clip shafts become more linear and the clips are moved radially inward toward each other (as depicted in FIG. 7). The outward flare of the clip shafts and the resulting distance between the clips can be controlled by the distal positioning of the delivery sheath or collar on the non-linear portions of the clip shafts.

In order to ensure proper orientation of the clip shafts and clips, in some embodiments the side of the clip shaft that is flattened or faceted is on the same side of the clip shaft as the clips (instead of the opposite side in the previous FIGs). Accordingly, the flattened or faceted surfaces of the clip shafts can be abutted against each other, to result in the crossed arrangement as shown.

Also, in this example, as best seen in FIG. 7 the distal end of the coupling collar has a funnel or umbrella that flares out over the atrial surface of the cardiac valve as the coupling collar moves toward the cardiac valve (toward the clips). In some embodiments, this umbrella is constructed of a wire framework (e.g., shape-set Nitinol, stainless steel, etc.) with a flexible impermeable membrane covering material (e.g., ePTFE, PTFE, polyester, Dacron, metallic foil, etc.) attached thereto. The wire framework can be shape-set to naturally flare radially outward (as shown in FIG. 7) when unconstrained by the sheath.

The umbrella can serve multiple purposes. In some cases, the umbrella acts as an occluder to mitigate retrograde flow (valvular regurgitation) of blood through the valve leaflets that are clipped together. Alternatively or additionally, in some cases the umbrella acts as a funnel for retrieving the clips into a sheath in the event that recapture and removal of the clips is desired.

In some embodiments, the umbrella has a circular cross-sectional shape. In some embodiments, the umbrella has an oblong cross-sectional shape (e.g., an oval, ellipse, egg-shaped, D-shaped, and the like). In some such embodiments, the major axis of the oblong cross-sectional shape can be orientated to extend along the line of coaptation of the native valve leaflets. Accordingly, the above-described occlusive performance of the umbrella can be optimized in such an orientation.

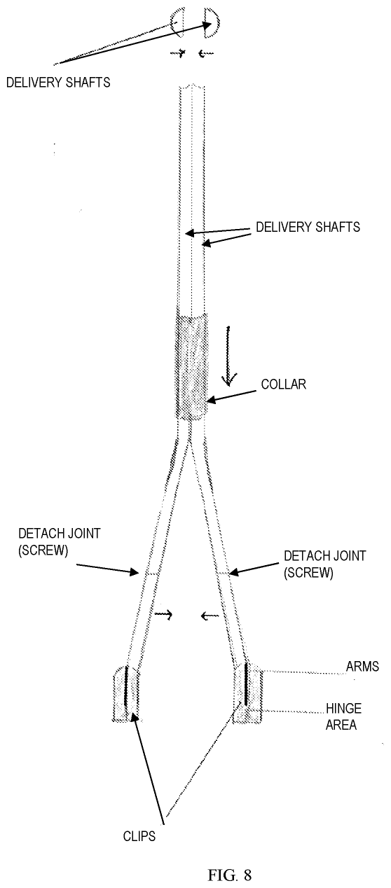

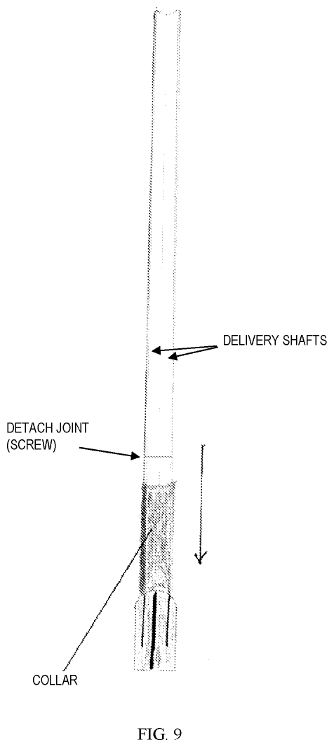

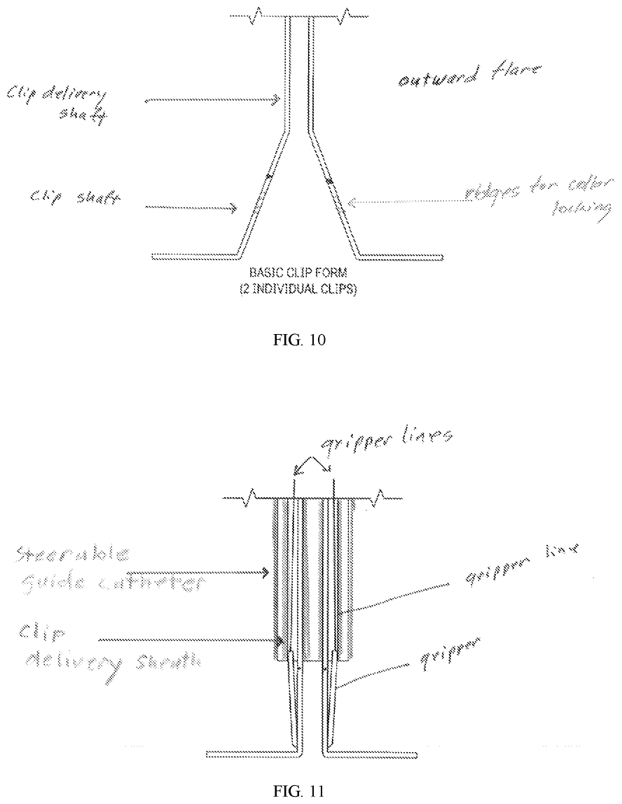

FIGS. 8 and 9 illustrate another example embodiment and sequence of joining two clips in a parallel configuration. The clip shafts in this embodiment also have a naturally curved or angled shape (as do the clip shafts of the embodiment described in above in reference to FIGS. 6 and 7). However, this embodiment is different than that of FIGS. 6 and 7 in that the clip shafts are shaped to not cross each other. Instead of crossing each other, each clip shaft is curved/angled to extend radially away from the other clips shaft. The embodiment of FIGS. 8 and 9 includes the collar and can include an umbrella attached at a distal end of the collar.

Again, the resting configuration of the clips is with the arms closed against each other. Compared to the previous FIGs, the arms are angled up at less than a 90-degree angle from the shaft of the clip. This resting configuration may improve tissue bonding.

After the step illustrated in FIG. 7 or in FIG. 9, the delivery shafts (clip shafts) are disconnected from the clips and removed/retrieved.

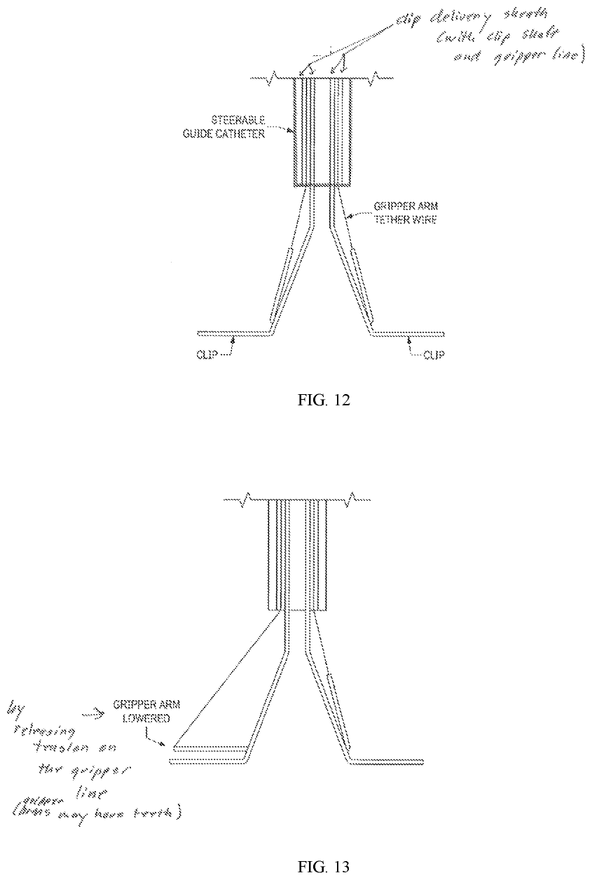

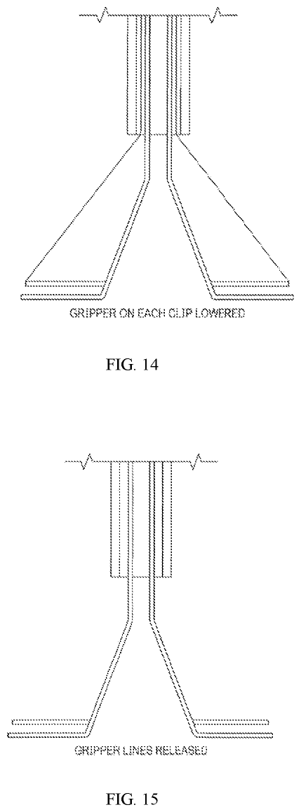

FIGS. 11-18 illustrate a sequence of the deployment of an implant. Unlike in the previous FIGs., both clips are housed together in a single small bore steerable guide (or catheter). The small bore steerable guide is not retrieved before the delivery of the collar, and the collar slides in it instead of in the large bore catheter.

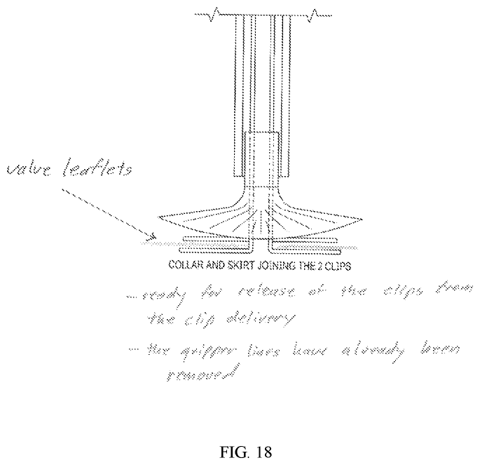

In FIGS. 11-18, all steerable guides are not shown, and additional steerable guides could be placed further up the FIGs. For simplicity, the valve leaflets are only shown in FIG. 18.

In FIG. 11, the clip delivery sheath houses the clip delivery shaft, which is detachable from the clip, and the tether wire or gripper line.

In FIG. 15, the gripper lines or tether wires are released, and the delivery sheaths are retrieved.

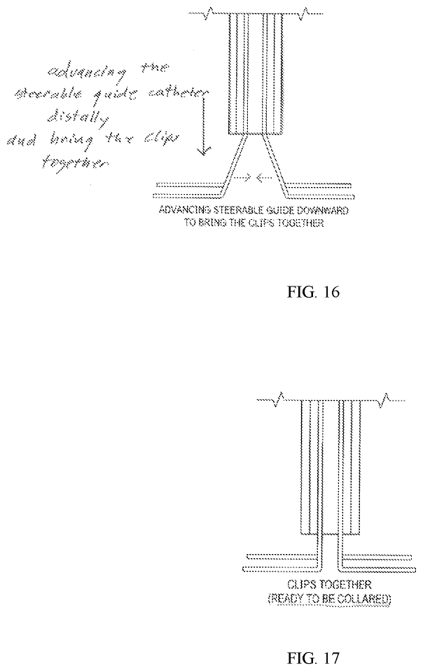

In FIGS. 16 and 17, the outward flare of the clip delivery shafts and the distance between the clips can be controlled by how far up or down the distal end of the small bore steerable guide is on the flared section of the clip delivery shaft and/or the clip shaft itself. The outward flare may be provided just on the clip delivery shaft (as illustrated), just on the clip shaft, or on both the clip shaft and the delivery shaft.

In FIG. 18, the collar allows for some rotation of the clips themselves. The clip shaft is rounded, optionally with horizontal ridges that loosely lock in with the horizontal ridges inside the collar lumens.

Additionally, the disclosure also contemplates at least the following embodiments:

### Embodiment 1

Embodiment 1 is an implant for reapproximating leaflets of a cardiac valve of a patient. The implant comprises two or more clips and a coupling collar.

Each clip comprises a shaft, a main arm, and a gripper arm. The main arm and/or the gripper arm are attached to the shaft via a hinge area. The gripper arm is located above the main arm on the same side of the shaft as the main arm. Each clip can be moved independently from the other clips and grasp a valve leaflet.

The coupling collar (e.g., a stent, a tube) is movable along the clips toward the main arm/gripper arm so as to join the two or more clips. The coupling collar is capable of joining the two or more clips.

### Embodiment 2

Embodiment 2 is an implant as described in embodiment 1 wherein each clip comprises a single main arm and a single gripper arm.

### Embodiment 3

Embodiment 3 is an implant as described in embodiments 1 or 2 wherein locking grooves or ridges are present on the proximal end of each shaft. These locking grooves or ridges can secure the coupling collar at the proper level, joining the two or more clips.

### Embodiment 4

Embodiment 4 is an implant as described in any of embodiments 1 to 3 wherein the shaft of each clip is flattened or faceted on the side opposite of the main arm and gripper arm so that the two or more clips can be joined in a parallel configuration.

### Embodiment 5

Embodiment 5 is an implant as described in any of embodiments 1 to 3 wherein the shaft of each clip is flattened or faceted on the same side as the main arm and gripper arm so that the two or more clips can be joined in a crossed configuration.

### Embodiment 6

Embodiment 6 is an implant as described in any of embodiments 1 to 5 wherein the shaft of each clip can rotate inside the coupling collar even when the coupling collar has been moved in place to interlock with the locking grooves or ridges on each shaft.

### Embodiment 7

Embodiment 7 is an implant as described in any of embodiments 1 to 6 wherein the coupling collar includes two portions releasably locked together.

### Embodiment 8

Embodiment 8 is a kit for delivering an implant as described in any of embodiments 1 to 7. The kit comprises two or more sheaths, each including a delivery shaft and a tether wire. The delivery shaft of each sheath is capable of being connected to the shaft of a clip. The tether wire is looped in a passageway in the sheath. The tether wire is capable of being releasably attached to the gripper arm of a clip and of displacing the gripper arm.

### Embodiment 9

Embodiment 9 is a kit as described in embodiment 8, further comprising two or more steerable guides, each capable of housing a sheath having a clip connected thereto.

### Embodiment 10

Embodiment 10 is a kit as described in embodiment 8, further comprising at least one steerable guide, the steerable guide being capable of housing two or more sheaths.

### Embodiment 11

Embodiment 11 is a kit as described in any of embodiments 8 to 10, wherein the delivery sheaths and/or the delivery shafts have a slight outward angle leading to an outward flare of the clips.

### Embodiment 12

Embodiment 12 is a kit as described in any of embodiments 8 to 11, further comprising a steerable catheter capable of housing the steerable guides.

### Embodiment 13

Embodiment 13 is a kit as described in any of embodiments 8 to 12, further comprising a collar delivery catheter capable of holding the coupling collar and sized to surround the two or more delivery shafts.

### Embodiment 14

Embodiment 14 is a method of implanting an implant in the heart of a patient as described in any of embodiments 1 to 7 using a kit as described in any of embodiments 8 to 13, wherein each clip can be moved independently from the other clips and grasp a valve leaflet. Once a proper grasp of the leaflets is completed with all the clips, the clips can be joined together, thus forming a single structure.

### Embodiment 15

Embodiment 15 is a method as described in embodiment 14, wherein pulling the clips together with the coupling collar causes the valve leaflets to be reapproximated.

### Embodiment 16

Embodiment 16 is a method as described in embodiments 14 or 15, further comprising allowing the shaft or each clip to rotate inside the coupling collar when the coupling collar interlock with the locking grooves or ridges on each shaft.

What we claim is:

1. An implantable heart valve leaflet clip system comprising:
a delivery sheath defining a lumen;
a first clip device slidably disposed within the lumen, the first clip device comprising a first shaft and a first leaflet clip releasably coupled to a distal end of the first shaft;
a second clip device slidably disposed within the lumen, the second clip device comprising a first shaft and a first leaflet clip releasably coupled to a distal end of the first shaft; and
a collar that is slidably disposed within the lumen and that is slidably disposed around the first and second shafts.

2. The system of claim 1, wherein the collar comprises an expandable umbrella portion having a naturally flared shape that is revealed when the collar is distally expressed from the lumen.

3. The system of claim 2, wherein the flared shape has a circular cross-sectional shape.

4. The system of claim 2, wherein the flared shape has an oblong cross-sectional shape.

5. The system of claim 4, wherein the oblong cross-sectional shape is elliptical or oval shaped.

6. The system of claim 2, wherein the umbrella portion comprises a flexible membranous material attached to a shape-set metal frame.

7. The system of claim 1, wherein the first and second shafts each have a naturally non-linear, curved, or angled shape by which the clips are disposed radially outward from the first and second shafts when the first and second shafts are radially unconstrained by the delivery sheath and the collar.

8. The system of claim 7, wherein the first and second shafts cross each other.

9. The system of claim 8, wherein the first and second shafts have abutting flat surfaces.

10. The system of claim 7, wherein the first and second shafts do not cross each other.

11. The system of claim 10, wherein the first and second shafts have abutting flat surfaces.

12. The system of claim 1, wherein the first and second leaflet clips are releasably coupled to the first and second shafts, respectively, by a threaded connection.

13. The system of claim 1, wherein the first and second leaflet clips are releasably coupled to the first and second shafts, respectively, by removable sutures.

14. The system of claim 1, wherein the first and second shafts are tubes.

15. The system of claim 1, wherein the system is configured such that positioning a distal tip of the delivery sheath or the collar at various positions along the first and second shafts adjustably controls a distance between the first and second clips.
